(19) United States
(12) Patent Application Publication
(10) Pub. No.: US 2009/0068210 A1
(43) Pub. Date: Mar. 12, 2009

(54) IMMUNOTHERAPY FOR HEMATOLOGICAL MALIGNANCIES

(76) Inventors: Nikhil C. Munshi, Boston, MA (US); Weihua Song, Boston, MA (US); Mark A. Exley, Boston, MA (US)

Correspondence Address: JONES DAY, 222 EAST 41ST ST, NEW YORK, NY, 10017, US

(21) Appl. No.: 12/169,585

(22) Filed: Jul. 8, 2008

Related U.S. Application Data
(60) Provisional application No. 60/958,875, filed on Jul. 9, 2007.

(51) Int. Cl. A61K 39/00; C12N 5/06; A61K 45/00

(52) U.S. Cl. 424/184.1; 435/375; 424/93.7

(57) ABSTRACT

Methods and compositions for treating, preventing and/or managing various hematological malignancies are disclosed. Specific methods and compositions relate to use of tumor cells engineered to express antigen presenting molecules which present antigens recognized by iNKT cells and eliciting antitumor immune response from iNKT cells using such tumor cells, optionally in combination with an immunomodulatory compound.

This application claims priority to U.S. Provisional No. 60/958,875, filed Jul. 9, 2007, which is incorporated herein by reference in its entirety.

### 1. FIELD OF THE INVENTION

Provided herein are immunotherapeutic methods for hematological malignancies such as hematological cancers, e.g., multiple myeloma. Pharmaceutical compositions and dosing regimens for such immunotherapy are also disclosed.

### 2. BACKGROUND OF THE INVENTION

### 2.1 Hematological Malignancies

Hematological malignancies are cancers of the body's blood-forming and immune systems—the bone marrow and lymphatic tissues. They include, e.g., leukemias, lymphomas, and myeloma. New cases of leukemia, lymphoma, and myeloma account for 9 percent of cancer cases diagnosed in the United States, and about 59,200 persons are killed by the diseases each year.

Leukemia is a disease characterized by a progressive and abnormal accumulation of white blood cells, or leukocytes. Abnormal accumulation of leukemic cells results in the organs and tissues being infiltrated by these cells. Bloodstream and bone marrow, where the accumulated cells disrupt the production of normal cells, are particularly susceptible. The resulting symptoms include fatigue, pallor, infections, bruising, and bleeding, and discomfort caused by enlarged organs. Currently available treatments include chemotherapy and bone marrow transplantation.

Lymphoma refers to any of a group of malignant neoplasms derived from cells endogenous to lymphoid tissue. Lymphomas include Hodgkin's disease (Hodgkin's lymphoma) and non-Hodgkin's lymphomas. The etiology of most lymphomas is unknown, although certain types of lymphomas were reported to be caused by viruses. Based on their immunological characteristics, lymphomas can further be classified into B-cell, T-cell, or M-cell type. Currently available treatments include chemotherapy and/or radiation.

Multiple myeloma, also known as plasma cell myeloma, is a common cancer of the blood. Multiple myeloma accounts for approximately 1 percent of all cancers and 2 percent of all deaths from cancer. Multiple myeloma is a disease in which malignant plasma cells spread through the bone marrow and hard outer portions of the large bones of the body. Eventually, multiple soft spots or holes, which are called osteolytic lesions, form in the bones in about 70 percent of individuals with multiple myeloma. As a result, osteoporosis may develop. Currently available treatments include chemotherapy, radiation, and/or blood transfusion.

The incidence of cancer in general, and hematological malignancies specifically, continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer including hematological malignancies. Thus, there continues to be a need for safe and effective methods of treating, preventing and managing cancer including hematological malignancies.

### 2.2 iNKT Cells

Invariant Natural Killer T (“iNKT”) cells are T lymphocytes that behave similarly to cells of the innate immune system. They recognize glycolipid antigens presented by the MHC class I-related protein CD1d. For example, studies from murine tumor models demonstrate that, upon activation by the glycolipid α-galactosylceramide (“α-GalCer”), which is a specific ligand for CDid, iNKT cells can stimulate potent antitumor immune response through production of Th-1 type cytokines such as IFN-γ.

While CD1d-restricted iNKT cells may play an important regulatory role in antitumor immune responses through the production of Th-1-type cytokines, the iNKT population is decreased in numbers, and in capacity to produce IFN-γ, in certain hematological malignancies and solid tumors. Therefore, the development of effective iNKT cells may provide an important avenue for an effective immunotherapy in various hematological malignancies.

### 2.3 Immunomodulatory Compounds

Immunomodulatory compounds provided herein, including the compounds referred to as IMiDs™ (Celgene Corporation), show potent inhibition of TNF-α but also marked inhibition of LPS induced monocyte IL1β and IL12 production. LPS induced IL6 is also inhibited by immunomodulatory compounds, albeit partially. These compounds are potent stimulators of LPS induced IL10. Particular examples of immunomodulatory compounds include, but are not limited to, the substituted 2-(2,6-dioxopiperidin-3-yl) phthalimides and substituted 2-(2,6-dioxopiperidin-3-yl)-1-oxoisoindoles described in U.S. Pat. Nos. 6,281,230 and 6,316,471, both to G. W. Muller, et al.

### 3. SUMMARY OF THE INVENTION

Provided herein are methods and compositions for treating, preventing, and/or managing cancer, in particular, hematological malignancies or solid cancers.

In one embodiment, provided herein is a tumor cell engineered to express antigen presenting molecules (e.g., an exogenous antigen presenting molecule), which present antigens recognized by iNKT cells, e.g., CD1d. In one embodiment, the expression of CD1d by the tumor cell is achieved by transfecting CD1d cDNA into the cell. In another embodiment, the CD1d expressing tumor cell may be optionally “preloaded” with α-GalCer by culturing the CD1d cDNA transfected tumor cell in the presence of α-GalCer. Also provided herein is a method of treating, preventing, or managing hematological malignancies comprising administering into a subject (e.g., human) a tumor cell described above.

In another embodiment, provided herein is a method of enhancing Th-1 type immune response from iNKT cells in vitro, or in a patient having a hematological malignancy. In one embodiment, the enhancement of Th-1 type immune response is achieved using a tumor cell transfected with CD1d cDNA. In another embodiment, the response is achieved using a tumor cell transfected with CD1d cDNA, optionally cultured in the presence of α-GalCer. In another embodiment, an increased enhancement may be achieved by optionally adding (in vitro), or co-administering to the patient, an immunomodulatory compound.

Pharmaceutical compositions, dosage forms, and kits used in connection with the methods described herein are also provided.

### 4. BRIEF DESCRIPTION OF THE FIGURES

FIG. 1A shows the purity of iNKT cells (pre-expansion) from a healthy donor, analyzed by flow cytometry using double staining with anti-Vα24-FITC and anti-Vβ11-PE mAbs.

FIG. 1B shows the purity of iNKT cells (post-expansion) from a healthy donor, analyzed by flow cytometry using double staining with anti-Vα24-FITC and anti-Vβ11-PE mAbs.

FIG. 1C shows the purity of iNKT cells (pre-expansion) from an advanced multiple myeloma patient, analyzed by flow cytometry using double staining with anti-Vα24-FITC and anti-Vβ11-PE mAbs.

FIG. 1D shows the purity of iNKT cells (post-expansion) from an advanced multiple myeloma patient, analyzed by flow cytometry using double staining with anti-Vα24-FITC and anti-Vβ11-PE mAbs.

FIG. 2A illustrates CD1d expression by normal plasma cells, multiple myeloma cell lines, and cells from multiple myeloma patients, as assessed by microarray analysis.

FIG. 2B illustrates CD1d expression in primary myeloma cells as assessed by flow cytometry analysis stained with PE-conjugated anti-CD1d mAb.

FIG. 2C illustrates CD1d expression of CD1d transfected MM.1S cell line as assessed by flow cytometry stained with PE-conjugated anti-CD1d mAb.

FIG. 3A illustrates the activation of iNKT cells cultured in the presence of α-GalCer alone, as assessed by double staining with anti-TCRVα24-FITC and anti-CD25-PC5 mAbs after 48 hours of incubation.

FIG. 3B illustrates the activation of iNKT cells cultured in the presence of CD1d positive primary myeloma cells, as assessed by double staining with anti-TCRVα24-FITC and anti-CD25-PC5 mAbs after 48 hours of incubation.

FIG. 3C illustrates the activation of iNKT cells cultured in the presence of CD1d positive primary myeloma cells pulsed with 100 ng/ml of α-GalCer, as assessed by double staining with anti-TCRVα24-FITC and anti-CD25-PC5 mAbs after 48 hours of incubation.

FIG. 3D shows the statistical analysis of CD25 expression by Vα24⁺ iNKT cells cultured in the presence of α-GalCer alone, CD1d positive primary myeloma cells, or CD1d positive primary myeloma cells pulsed with α-GalCer (100 ng/ml, n=5).

FIG. 4A illustrates IFN-γ and IL-4 production by iNKT cells incubated with CD1d expressing multiple myeloma cells or CD1d expressing multiple myeloma cells pulsed with α-GalCer. Levels of IFN-γ and IL-4 were determined by ELISA at 72 hours and 48 hours post-treatment, respectively.

FIG. 4B illustrates IL-2 production by iNKT cells incubated with CD1d expressing multiple myeloma cells or CD1d expressing multiple myeloma cells pulsed with α-GalCer. Level of IL-2 was determined by ELISA at 72 hours post-treatment.

FIG. 4C illustrates IFN-γ and IL-4 production by iNKT cells incubated with dendritic cells (“DC”) or DCs pulsed with α-GalCer. Levels of IFN-γ and IL-4 were determined by ELISA at 72 hours and 48 hours post-treatment, respectively.

FIG. 4D illustrates IL-2 production by iNKT cells incubated with DCs or DCs pulsed with α-GalCer. Level of IL-2 was determined by ELISA at 72 hours post-treatment.

FIG. 5A shows the increase in IFN-γ levels where iNKT cells from healthy donors and multiple myeloma patients were treated with CD1d expressing multiple myeloma cells in combination with 1-oxo-2-(2,6-dioxopiperidin-3-yl)-4-aminoisoindoline, as compared with the treatment with CD1d expressing multiple myeloma cells alone.

FIG. 5B shows the increase in IL-2 levels where iNKT cells from healthy donors and multiple myeloma patients were treated with CD1d expressing multiple myeloma cells in combination with 1-oxo-2-(2,6-dioxopiperidin-3-yl)-4-aminoisoindoline, as compared with the treatment with CD1d expressing multiple myeloma cells alone.

FIG. 5C shows the decrease in IL-4 levels where iNKT cells from healthy donors and multiple myeloma patients were treated with CD1d expressing multiple myeloma cells in combination with 1-oxo-2-(2,6-dioxopiperidin-3-yl)-4-aminoisoindoline, as compared with the treatment with CD1d expressing multiple myeloma cells alone.

FIG. 5D shows the increase in IFN-γ levels where iNKT cells from healthy donors and multiple myeloma patients were treated with CD1d expressing multiple myeloma cells, pulsed with α-GalCer, in combination with 1-oxo-2-(2,6-dioxopiperidin-3-yl)-4-aminoisoindoline, as compared with the treatment with CD1d expressing multiple myeloma cells pulsed with α-GalCer alone.

FIG. 5E shows the increase in IL-2 levels where iNKT cells from healthy donors and multiple myeloma patients were treated with CD1d expressing multiple myeloma cells, pulsed with α-GalCer, in combination with 1-oxo-2-(2,6-dioxopiperidin-3-yl)-4-aminoisoindoline, as compared with the treatment with CD1d expressing multiple myeloma cells pulsed with α-GalCer alone.

FIG. 5F shows the decrease in IL-4 levels where iNKT cells from healthy donors and multiple myeloma patients were treated with CD1d expressing multiple myeloma cells, pulsed with α-GalCer, in combination with 1-oxo-2-(2,6-dioxopiperidin-3-yl)-4-aminoisoindoline, as compared with the treatment with CD1d expressing multiple myeloma cells pulsed with α-GalCer alone.

### 5. DETAILED DESCRIPTION OF THE INVENTION

Provided herein are compositions and methods for treating, preventing, and/or managing hematological malignancies, e.g., solid tumors. In one embodiment, provided herein is a tumor cell which expresses antigen presenting molecules, which present antigens recognized by iNKT cells. In one embodiment, such antigen presenting molecules are exogenous, i.e., are not naturally expressed in the tumor cell or have low level of expression in the tumor cell. Examples of such antigen presenting molecules include, but are not limited to, CD1d. In one embodiment, said tumor cell can be made by, for example, transfecting the tumor cell with CD1d cDNA. In some embodiments, said tumor cell may be “preloaded,” e.g., cultured in presence of the ligands recognized by the antigen presenting molecule expressed in the tumor cell. In one specific embodiment, CD1d expressing tumor cells may be preloaded with α-GalCer. Examples of tumor cells that may be used in connection with methods and compositions provided herein are described in detail in Section 5.2 below.

In another embodiment, provided herein is a method of enhancing Th-1 type immune response from iNKT cells in vitro, or in a patient having a hematological malignancy. In one embodiment, the enhancement of Th-1 type immune response is achieved using a tumor cell described above. In one embodiment where the treatment of a patient is contemplated, the tumor cell is preferably autologous. In another embodiment, the response is achieved using a tumor cell transfected with CD1d cDNA, optionally cultured in the presence of α-GalCer. In another embodiment, an increased enhancement may be achieved by optionally adding (in vitro), or co-administering to the patient, an immunomodulatory compound. Examples of immunomodulatory compounds are described in detail in Section 5.3 below. Doses and dosing regimens in connection with these methods are also provided herein.

In some embodiments, provided herein are pharmaceutical compositions, dosage forms, and kits comprising said tumor cells described above (with or without preloading with the ligands), and optionally an immunomodulatory compound. Such compositions and dosage forms may comprise additional active agents.

### 5.1 DEFINITIONS

As used herein, and unless otherwise specified, the term “enhancing Th-1 type immune response” means that, when a tumor cell provided herein is added (in vitro) or administered to a subject, with or without an immunomodulatory compound, there is an increase in levels of cytokines associated with Th-1 type immunity, measured using any standard methods known in the art and described herein, as compared to where no such addition or administration by said tumor cell is made. Examples of cytokines associated with Th-1 type immunity include, but are not limited to, IFN-γ and IL-2. In one embodiment, an enhanced Th-1 immune response means about 5%, 10%, 20%, 30%, 50%, 70%, 80%, or 100% or greater increase in Th-1 cytokine levels.

Alternatively, the term “enhancing Th-1 type immune response” means that, when a tumor cell provided herein is added (in vitro) or administered to a subject, with or without an immunomodulatory compound, a low level of cytokines associated with Th-2 type immunity, measured using any standard methods known in the art and described herein, is released. Examples of cytokines associated with Th-2 type immunity include, but are not limited to, IL-4.

As used herein, and unless otherwise specified, the term “loading” or “preloading,” where used in connection with tumor cells, means the tumor cells are co-cultured with a molecule with which the tumor cells are preloaded.

As used herein, and unless otherwise specified, the term “autologous” means that a molecule referred to is derived from the same subject's body, e.g., a patient.

As used herein, and unless otherwise specified, the term “hematological malignancy” refers to are cancer of the body's blood-forming and immune system—the bone marrow and lymphatic tissue. Such cancers include leukemias, lymphomas (Non-Hodgkin's Lymphoma), Hodgkin's disease (also called Hodgkin's Lymphoma) and myeloma.

As used herein, and unless otherwise specified, the term “adoptive transfer” refers to a form of passive immunization where previously sensitized immunologic agents (e.g., cells or serum) are transferred to non-immune recipients.

As used herein, and unless otherwise specified, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. Suitable are hydrochloric, hydrobromic, phosphoric, and sulfuric acids.

As used herein, and unless otherwise specified, the term “solvate” means a compound, or a salt thereof, that further includes a stoichiometric or non-stoichiometric amount of solvent bound by non-covalent intermolecular forces. Where the solvent is water, the solvate is a hydrate.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, and unless otherwise specified, the term “stereoisomer” encompasses all enantiomerically/stereomerically pure and enantiomerically/stereomerically enriched compounds disclosed herein.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90%, or 95% or more of one stereoisomer and 20%, 10%, or 5% or less of the counter stereoisomer. In certain cases, a compound provided herein is considered optically active or stereomerically/enantiomerically pure (i.e., substantially the R-form or substantially the S-form) with respect to a chiral center when the compound is about 80% ee (enantiomeric excess) or greater, preferably, equal to or greater than 90% ee with respect to a particular chiral center, and more preferably 95% ee with respect to a particular chiral center.

As used herein, and unless otherwise indicated, the term “stereomerically enriched” or “enantiomerically enriched” encompasses racemic mixtures as well as other mixtures of stereoisomers of compounds provided herein (e.g., R/S=30/70, 35/65, 40/60, 45/55, 55/45, 60/40, 65/35 and 70/30).

As used herein, and unless otherwise specified, the terms “treat,” “treating” and “treatment” contemplate an action that occurs while a patient is suffering from the specified disease or disorder, which reduces the severity of the disease or disorder, or retards or slows the progression of the disease or disorder.

As used herein, unless otherwise specified, the terms “prevent,” “preventing” and “prevention” contemplate an action that occurs before a patient begins to suffer from the specified disease or disorder, which inhibits or reduces the severity of the disease or disorder.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, and unless otherwise specified, the term “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, the term “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

### 5.2 TREATMENT, PREVENTION, AND MANAGEMENT OF HEMATOLOGICAL MALIGNANCIES

In some embodiments, provided herein are tumor cells engineered to express an antigen presenting molecule, which presents an antigen recognized by iNKT cells and methods of producing said tumor cells. Such tumor cells may be used in connection with the treatment or vaccination against various hematological malignancies. In certain specific embodiments, said tumor cells may be those engineered to express CD1d. CD1d-expressing tumor cells can be made by, for example, transfecting non-CD1d expressing tumor cells or CD1d expressing tumor cells (e.g., where there is a need for enhanced CD1d expression) with a cDNA which encodes CD1d, using methods known in the art.

Without being limited by a particular theory, it is believed that CD1d presents an antigen that is recognized by iNKT cells, thus, can enhance the immune response to the tumor cells bearing the molecule. Without limited by a particular theory, it is believed that tumor cells bearing CD1d can thus partially or fully rectify the impairment of iNKT cells, be it reduction in numbers or reduction in functionalities, and enhance the Th-1 type immune response to the tumor cells elicited from iNKT cells.

In some embodiments, it was discovered that the enhancement of the Th-1 type immune response is furthered where the CD1d expressing tumor cells are preloaded with, for example, α-GalCer, a ligand of CD1d. Accordingly, one embodiment provided herein is directed to tumor cells which expresses CD1d, and preloaded with α-GalCer.

Tumor cells from various hematological malignancies may be used in connection with the compositions and methods provided herein. Examples of hematological malignancies include, but are not limited to: leukemias including, but not limited to, chronic lymphocytic leukemia, chronic myelocytic leukemia, acute lymphoblastic leukemia (ALL), acute myelogenous leukemia (AML) and acute myeloblastic leukemia; lymphomas including, but are not limited to, Hodgkin's and non-Hodgkin's lymphomas, including all of the subtypes thereof; and myelomas including, but not limited to, multiple myeloma. In some embodiments, the hematological malignancy is multiple myeloma. Thus, one specific embodiment provided herein is directed to multiple myeloma cells which express CD1d, optionally preloaded with α-GalCer. Tumor cells engineered according to the description above may be altered (e.g., irradiated) in some cases.

Also provided herein are methods of enhancing Th-1 type immune response from iNKT cells (e.g., methods of activating iNKT cells) in vitro or in a subject, e.g., a human patient. In some embodiments, said methods comprise producing tumor cells engineered to express an antigen presenting molecule, which presents an antigen recognized by iNKT cells and administering (e.g., by adoptive transfer) the engineered tumor cells to a patient suffering from a hematological malignancy.

In some embodiments, it is preferred that the tumor cells are autologous, i.e., derived from the patient who receives the therapy. In some embodiments, the CD1d expressing tumor cells may be preloaded with α-GalCer. In some embodiments, the tumor cells are those obtained from a patient suffering from multiple myeloma and the hematological malignancy treated is multiple myeloma. In some embodiments, the CD1d expressing tumor cells are irradiated, preferably, before the preloading with α-GalCer.

In addition, it was surprisingly discovered that an immunomodulatory compound may further enhance the CD1d expressing tumor cells' ability to activate iNKT cells and to elicit Th-1 type immune response. It was also discovered that an immunomodulatory compound may enhance the CD1d expressing tumor cells' ability to activate iNKT cells and to elicit Th-1 type immune response with or without the preloading of the tumor cells with α-GalCer.

Thus, certain embodiments provided herein are directed to methods of enhancing Th-1 type immune response in a subject, e.g., human, comprising: producing a tumor cell expressing an antigen presenting molecule which presents an antigen recognized by iNKT cells; optionally irradiating said tumor cell; optionally preloading said tumor cell with a ligand for said antigen presenting molecule; and administering (e.g., by adoptive transfer) said tumor cell, in combination with an immunomodulatory compound, to a patient.

In a further embodiment, provided herein is a method of providing highly purified iNKT cells from a patient (i.e., autologous) suffering from, or likely to suffer from, a hematological malignancy, e.g., multiple myeloma. The methods comprise: 1) isolation of iNKT cells from a patient suffering from, or likely to suffer from, a hematological malignancy; and 2) incubating said iNKT cells with an immunomodulatory compound provided herein. In one embodiment, the iNKT cells obtained from a patient may be stimulated with a tumor cell expressing an antigen presenting molecule which presents an antigen recognized by the iNKT cells, e.g., α-GalCer loaded multiple myeloma cell. In one embodiment, the iNKT cells are TCRVα24⁺ and/or TCRVβ11⁺ iNKT cells.

In one embodiment, provided herein are compositions comprising the iNKT cells prepared by methods described herein. Methods of treating a hematological malignancy using such iNKT cells are also encompassed. In certain embodiments, the iNKT cells prepared according to methods described herein may be made into vaccines for the hematological malignancies provided herein. Where vaccines are made from the iNKT cells prepared as described herein, the cells may be irradiated before the administration (e.g., injection) to a patient.

In another embodiment, iNKT cells prepared as described herein may be stimulated, in vitro or in vivo, by an immunomodulatory compound. For example, iNKT cells, prepared and irradiated, may be administered to a patient with a hematological malignancy, in combination with an immunomodulatory compound. An immunomodulatory compound may be administered prior to, concurrently with, or subsequent to the administration of the iNKT cells. In one embodiment, an immunomodulatory compound is administered subsequent to the administration of the iNKT cells.

In one embodiment, the tumor cell is multiple myeloma cell. In one embodiment, the antigen presenting molecule is CD1d, and the ligand is α-GalCer. In one embodiment, the immunomodulatory compound is 1-oxo-2-(2,6-dioxopiperidin-3-yl)-4-aminoisoindoline or 1,3-dioxo-2-(2,6-dioxopiperidin-3-yl)-4-aminoisoindoline.

In one embodiment, the immunomodulatory compound may be administered prior to the administration of said tumor cells. In another embodiment, the immunomodulatory compound may be administered simultaneously said tumor cells. In another embodiment, the immunomodulatory compound may be administered subsequent to the administration of said tumor cells

In one embodiment, an immunomodulatory compound can be administered orally and in single or divided daily doses in an amount of from about 0.10 to about 150 mg/day. In one embodiment, 1,3-dioxo-2-(2,6-dioxopiperidin-3-yl)-4-aminoisoindoline may be administered in an amount of from about 0.1 to about 1 mg per day, or from about 0.1 to about 5 mg every other day. In another embodiment, 1-oxo-2-(2,6-dioxopiperidin-3-yl)-4-aminoisoindoline may be administered in an amount of from about 1 to about 25 mg per day, or from about 10 to about 50 mg every other day. In another embodiment, 1-oxo-2-(2,6-dioxopiperidin-3-yl)-4-aminoisoindoline may be administered in an amount of about 50 mg per day. In another embodiment, 1-oxo-2-(2,6-dioxopiperidin-3-yl)-4-aminoisoindoline may be administered in an amount of about 25 mg per day. In another embodiment, 1-oxo-2-(2,6-dioxopiperidin-3-yl)-4-aminoisoindoline may be administered in an amount of about 10 mg per day.

All of the methods provided herein may be practiced in combination with any known anti-cancer therapies such as, but not limited to, chemotherapies and radiation. Additional anti-cancer agents that may be used in combination with methods and compositions provided herein are described in detail in Section 5.4.5, below.

### 5.3 IMMUNOMODULATORY COMPOUNDS

Immunomodulatory compounds can either be commercially purchased or prepared according to the methods described in the patents or patent publications referred to herein, all of which are incorporated by reference. Further, optically pure compositions can be asymmetrically synthesized or resolved using known resolving agents or chiral columns as well as other standard synthetic organic chemistry techniques. Immunomodulatory compounds may be racemic, stereomerically enriched or stereomerically pure, and may encompass pharmaceutically acceptable salts, solvates, and prodrugs thereof.

As used herein and unless otherwise indicated, the terms “immunomodulatory compounds” encompass small organic molecules that markedly inhibit TNF-α, LPS induced monocyte IL-1β and IL-12, and partially inhibit IL-6 production. Particular examples include those referred to as IMiDs™ (Celgene Corporation).

Specific examples of immunomodulatory compounds, include, but are not limited to, cyano and carboxy derivatives of substituted styrenes such as those disclosed in U.S. Pat. No. 5,929,117; 1-oxo-2-(2,6-dioxo-3-fluoropiperidin-3-yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperidin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), including, but not limited to, those disclosed in U.S. Pat. Nos. 5,635,517, 6,476,052, 6,555,554, and 6,403,613; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; and isoindole-imide compounds such as those described in U.S. patent publication no. 2003/0045552 A1, U.S. Pat. No. 7,091,353, and WO 02/059106. The entireties of each of the patents and patent applications identified herein are incorporated herein by reference. Immunomodulatory compounds do not include thalidomide.

Other specific immunomodulatory compounds include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference. These compounds have the structure I:

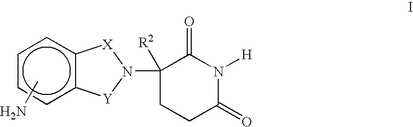

in which one of X and Y is C═O, the other of X and Y is C═O or CH₂, and R² is hydrogen or lower alkyl, in particular methyl. Specific immunomodulatory compounds include, but are not limited to:
1-oxo-2-(2,6-dioxopiperidin-3-yl)-4-aminoisoindoline;
1-oxo-2-(2,6-dioxopiperidin-3-yl)-5-aminoisoindoline;
1-oxo-2-(2,6-dioxopiperidin-3-yl)-6-aminoisoindoline;
1-oxo-2-(2,6-dioxopiperidin-3-yl)-7-aminoisoindoline;
1,3-dioxo-2-(2,6-dioxopiperidin-3-yl)-4-aminoisoindoline; and
1,3-dioxo-2-(2,6-dioxopiperidin-3-yl)-5-aminoisoindoline.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and WO 98/03502, each of which is incorporated herein by reference. Representative compounds are of formula:

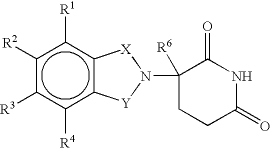

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R²,

R³ and R⁴ is fluoro or (ii) one of R¹, R², R³, or R⁴ is amino.

Compounds representative of this class are of the formulas:

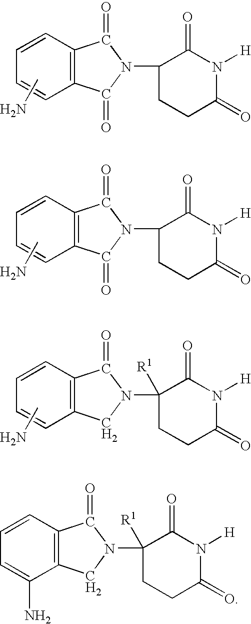

wherein R¹ is hydrogen or methyl. In a separate embodiment, encompassed is the use of enantiomerically pure forms (e.g. optically pure (R) or (S) enantiomers) of these compounds.

Still other specific immunomodulatory compounds belong to a class of isoindole-imides disclosed in U.S. Patent Application Publication Nos. US 2003/0096841 and US 2003/0045552, and WO 02/059106, each of which are incorporated herein by reference. Representative compounds are of formula II:

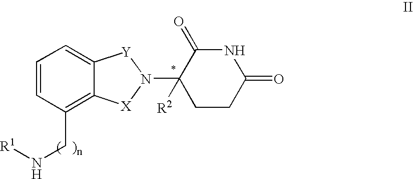

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl)-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

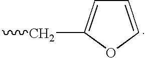

In another embodiment of the compounds of formula II, R¹ is

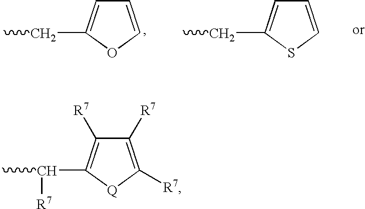

wherein Q is O or S, and each occurrence of R⁷ is independently H, (C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindoline-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl]methyl}(benzylamino)carboxamide.

Still other specific immunomodulatory compounds belong to a class of isoindole-imides disclosed in U.S. Patent Application Publication No. US 2002/0045643, International Publication No. WO 98/54170, and U.S. Pat. No. 6,395,754, each of which is incorporated herein by reference. Representative compounds are of formula III:

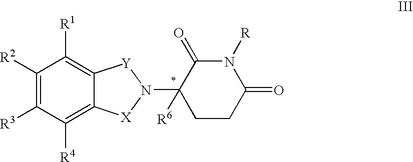

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center.

Other representative compounds are of formula:

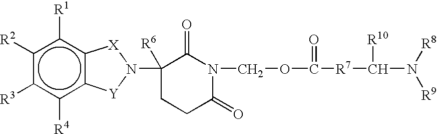

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂ X¹CH₂CH₂— in which X¹ is —O—, —S—, or —NH—;

R¹⁰ is hydrogen, alkyl of to 8 carbon atoms, or phenyl.

Other representative compounds are of formula:

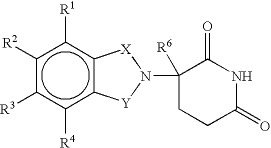

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

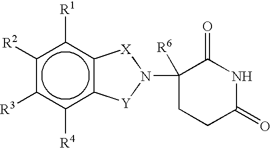

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

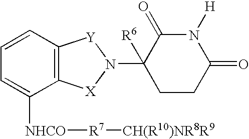

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

The most preferred immunomodulatory compounds are 4-(amino)-2-(2,6-dioxo(3-piperidyl))-isoindoline-1,3-dione and 3-(4-amino-1-oxo-1,3-dihydro-isoindol-2-yl)-piperidine-2,6-dione. The compounds can be obtained via standard, synthetic methods (see e.g., U.S. Pat. No. 5,635,517, incorporated herein by reference). The compounds are available from Celgene Corporation, Warren, N.J. 4-(Amino)-2-(2,6-dioxo(3-piperidyl))-isoindoline-1,3-dione has the following chemical structure:

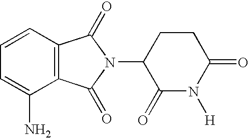

The compound 3-(4-amino-1-oxo-1,3-dihydro-isoindol-2-yl)-piperidine-2,6-dione has the following chemical structure:

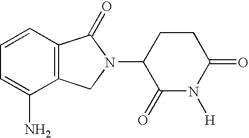

In another embodiment, specific immunomodulatory compounds encompass polymorphic forms of 3-(4-amino-1-oxo-1,3 dihydro-isoindol-2-yl)-piperidene-2,6-dione such as Form A, B, C, D, E, F, G and H, disclosed in U.S. publication no. US 2005/0096351 A1, which is incorporated herein by reference. For example, Form A of 3-(4-amino-1-oxo-1,3 dihydro-isoindol-2-yl)-piperidene-2,6-dione is an unsolvated, crystalline material that can be obtained from non-aqueous solvent systems. Form A has an X-ray powder diffraction pattern comprising significant peaks at approximately 8, 14.5, 16, 17.5, 20.5, 24 and 26 degrees 2θ, and has a differential scanning calorimetry melting temperature maximum of about 270° C. Form A is weakly or not hygroscopic and appears to be the most thermodynamically stable anhydrous polymorph of 3-(4-amino-1-oxo-1,3 dihydro-isoindol-2-yl)-piperidine-2,6-dione discovered thus far.

Form B of 3-(4-amino-1-oxo-1,3 dihydro-isoindol-2-yl)-piperidene-2,6-dione is a hemihydrated, crystalline material that can be obtained from various solvent systems, including, but not limited to, hexane, toluene, and water. Form B has an X-ray powder diffraction pattern comprising significant peaks at approximately 16, 18, 22 and 27 degrees 2θ, and has endotherms from DSC curve of about 146 and 268° C., which are identified dehydration and melting by hot stage microscopy experiments. Interconversion studies show that Form B converts to Form E in aqueous solvent systems, and converts to other forms in acetone and other anhydrous systems.

Form C of 3-(4-amino-1-oxo-1,3 dihydro-isoindol-2-yl)-piperidene-2,6-dione is a hemisolvated crystalline material that can be obtained from solvents such as, but not limited to, acetone. Form C has an X-ray powder diffraction pattern comprising significant peaks at approximately 15.5 and 25 degrees 2θ, and has a differential scanning calorimetry melting temperature maximum of about 269° C. Form C is not hygroscopic below about 85% RH, but can convert to Form B at higher relative humidities.

Form D of 3-(4-amino-1-oxo-1,3 dihydro-isoindol-2-yl)-piperidene-2,6-dione is a crystalline, solvated polymorph prepared from a mixture of acetonitrile and water. Form D has an X-ray powder diffraction pattern comprising significant peaks at approximately 27 and 28 degrees 2θ, and has a differential scanning calorimetry melting temperature maximum of about 270° C. Form D is either weakly or not hygroscopic, but will typically convert to Form B when stressed at higher relative humidities.

Form E of 3-(4-amino-1-oxo-1,3 dihydro-isoindol-2-yl)-piperidene-2,6-dione is a dihydrated, crystalline material that can be obtained by slurrying 3-(4-amino-1-oxo-1,3 dihydro-isoindol-2-yl)-piperidene-2,6-dione in water and by a slow evaporation of 3-(4-amino-1-oxo-1,3 dihydro-isoindol-2-yl)-piperidene-2,6-dione in a solvent system with a ratio of about 9:1 acetone:water. Form E has an X-ray powder diffraction pattern comprising significant peaks at approximately 20, 24.5 and 29 degrees 2θ, and has a differential scanning calorimetry melting temperature maximum of about 269° C. Form E can convert to Form C in an acetone solvent system and to Form G in a THF solvent system. In aqueous solvent systems, Form E appears to be the most stable form. Desolvation experiments performed on Form E show that upon heating at about 125° C. for about five minutes, Form E can convert to Form B. Upon heating at 175° C. for about five minutes, Form B can convert to Form F.

Form F of 3-(4-amino-1-oxo-1,3 dihydro-isoindol-2-yl)-piperidene-2,6-dione is an unsolvated, crystalline material that can be obtained from the dehydration of Form E. Form F has an X-ray powder diffraction pattern comprising significant peaks at approximately 19, 19.5 and 25 degrees 2θ, and has a differential scanning calorimetry melting temperature maximum of about 269° C.

Form G of 3-(4-amino-1-oxo-1,3 dihydro-isoindol-2-yl)-piperidene-2,6-dione is an unsolvated, crystalline material that can be obtained from slurrying forms B and E in a solvent such as, but not limited to, tetrahydrofuran (THF). Form G has an X-ray powder diffraction pattern comprising significant peaks at approximately 21, 23 and 24.5 degrees 2θ, and has a differential scanning calorimetry melting temperature maximum of about 267° C.

Form H of 3-(4-amino-1-oxo-1,3 dihydro-isoindol-2-yl)-piperidene-2,6-dione is a partially hydrated (about 0.25 moles) crystalline material that can be obtained by exposing Form E to 0% relative humidity. Form H has an X-ray powder diffraction pattern comprising significant peaks at approximately 15, 26 and 31 degrees 2θ, and has a differential scanning calorimetry melting temperature maximum of about 269° C.

Other specific immunomodulatory compounds include, but are not limited to, 1-oxo-2-(2,6-dioxo-3-fluoropiperidin-3-yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

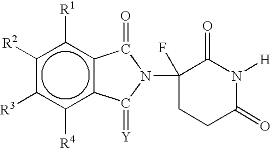

wherein Y is oxygen or H² and

each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds include, but are not limited to, the tetra substituted 2-(2,6-dioxopiperidin-3-yl)-1-oxoisoindolines described in U.S. Pat. No. 5,798,368, which is incorporated herein by reference. Representative compounds are of formula:

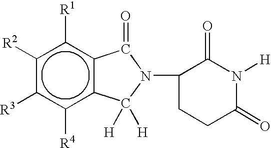

wherein each of R¹, R², R³, and R⁴, independently of the others, is halo, alkyl of 1 to 4 carbon atoms, or alkoxy of 1 to 4 carbon atoms.

Other specific immunomodulatory compounds include, but are not limited to, 1-oxo and 1,3-dioxo-2-(2,6-dioxopiperidin-3-yl) isoindolines disclosed in U.S. Pat. No. 6,403,613, which is incorporated herein by reference. Representative compounds are of formula:

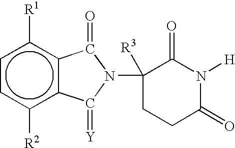

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

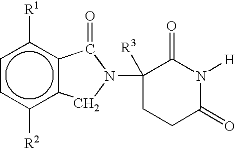

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

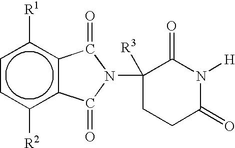

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl.

Other specific immunomodulatory compounds include, but are not limited to, 1-oxo and 1,3-dioxoisoindolines substituted in the 4- or 5-position of the indoline ring described in U.S. Pat. No. 6,380,239 and U.S. publication no. US 2006/0084815 A1, which are incorporated herein by reference. Representative compounds are of formula:

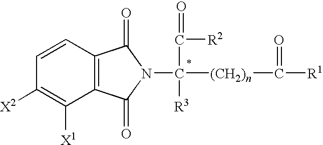

in which the carbon atom designated C* constitutes a center of chirality (when n is not zero and R¹ is not the same as R²); one of X¹ and X² is amino, nitro, alkyl of one to six carbons, or NH-Z, and the other of X¹ or X² is hydrogen; each of R¹ and R² independent of the other, is hydroxy or NH-Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

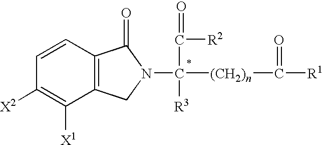

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

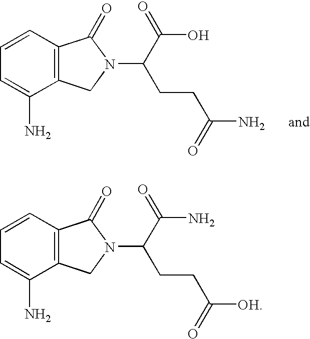

Other representative compounds are of formula:

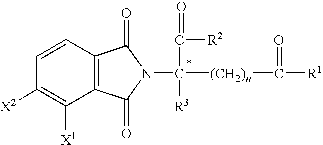

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

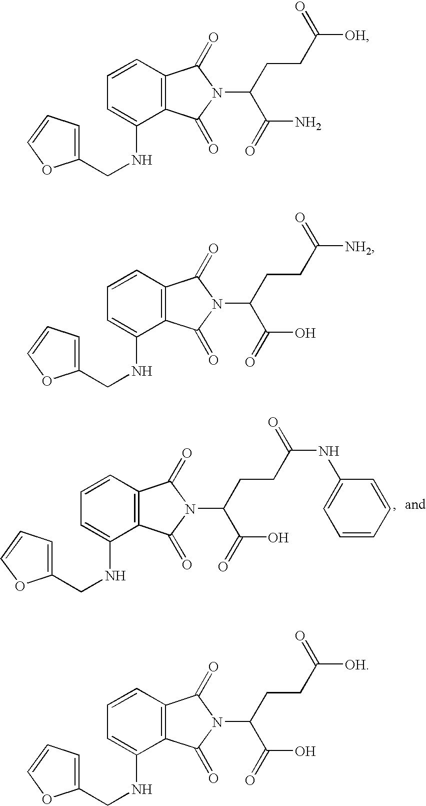

Other specific examples of the compounds are of formula:

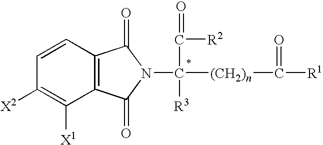

wherein one of X¹ and X² is nitro, or NH-Z, and the other of X¹ or X² is hydrogen;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2;

provided that if one of X¹ and X² is nitro, and n is 1 or 2, then R¹ and R² are other than hydroxy; and

if —COR² and —(CH₂)ₙCOR¹ are different, the carbon atom designated C* constitutes a center of chirality. Other representative compounds are of formula:

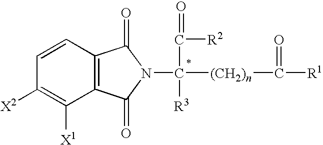

wherein one of X¹ and X² is alkyl of one to six carbons;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

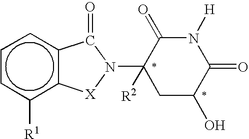

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen;

and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

Compounds provided herein can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

Various immunomodulatory compounds contain one or more chiral centers, and can exist as racemic mixtures of enantiomers or mixtures of diastereomers. Encompassed is the use of stereomerically pure forms of such compounds, as well as the use of mixtures of those forms. For example, mixtures comprising equal or unequal amounts of the enantiomers of a particular immunomodulatory compounds may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.4 PHARMACEUTICAL COMPOSITIONS AND DOSAGE FORMS

Pharmaceutical compositions can be used in the preparation of individual, single unit dosage forms. Pharmaceutical compositions and dosage forms provided herein comprise a tumor cell engineered to express an antigen presenting molecule which presents an antigen recognized by iNKT cells, and optionally an immunomodulatory compound, or a pharmaceutically acceptable salt, solvate, stereoisomer, or prodrug thereof. In one embodiment, the tumor cell may be preloaded with a ligand for said antigen presenting molecule. In some embodiments, pharmaceutical compositions and dosage forms may further comprise additional active agents. Pharmaceutical compositions and dosage forms provided herein can further comprise one or more excipients.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, encompassed are pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Particular lactose-free dosage forms comprise active ingredients, microcrystalline cellulose, pre-gelatinized starch, and magnesium stearate.

Also encompassed are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability. Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Also encompassed are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

The specific amount of the tumor cell used in the compositions and dosage forms provided herein will depend on the type of cancer being treated or managed, age and condition of the patient being treated, and the manner of treatment, and amount(s) of an immunomodulatory compound and any optional additional active agents concurrently administered to the patient, and may be properly determined by those skilled in the art.

In some embodiments, compositions and dosage forms provided herein comprise an immunomodulatory compound, or a pharmaceutically acceptable salt, solvate, stereoisomer, or prodrug thereof, in an amount of from about 0.10 to about 150 mg. In some embodiments, compositions and dosage forms provided herein comprise an immunomodulatory compound, or a pharmaceutically acceptable salt, solvate, stereoisomer, or prodrug thereof, in an amount of about 0.1, 1, 2, 5, 7.5, 10, 12.5, 15, 17.5, 20, 25, 50, 100, 150 or 200 mg.

In certain embodiments where the use of additional active ingredients is contemplated, compositions and dosage forms provided herein comprise the additional active ingredient in an amount of 1 to about 1000 mg, from about 5 to about 500 mg, from about 10 to about 350 mg, or from about 50 to about 200 mg. The specific amount of the agent will depend on the specific agent used, the type of disease or disorder being treated or managed, and the amount(s) of tumor cells, an immunomodulatory compound and any other optional additional active agents concurrently administered to the patient.

5.4.1 Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Oral dosage forms provided herein may be prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g. powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is typically present in from about 50 to about 99 weight percent of the pharmaceutical composition or dosage form.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.4.2 Delayed Release Dosage Forms

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients. Thus, encompassed are single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms. For example, cyclodextrin and its derivatives can be used to increase the solubility of an immunomodulatory compound and its derivatives. See, e.g., U.S. Pat. No. 5,134,127, which is incorporated herein by reference.

5.4.4 Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms provided herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.4.5 Additional Active Agents

In addition to compositions and methods described above, other pharmacologically active agents may be used in combination with methods and compositions provided herein. It is believed that certain combinations may work synergistically in the treatment of hematological malignancies, and conditions and symptoms associated with such malignancies. Compositions and methods provided herein can also work to alleviate adverse effects associated with certain additional active agents, and vice versa.

Examples of additional active agents include, but are not limited to: semaxanib; cyclosporin; etanercept; doxycycline; bortezomib; acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Other examples of active agents include, but are not limited to, 2-methoxyestradiol, telomestatin, inducers of apoptosis in multiple myeloma cells (such as, for example, TRAIL), statins, semaxanib, cyclosporin, etanercept, doxycycline, bortezomib, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, Tykerb® (lapatinib), methotrexate, Arisa®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, paclitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

5.4.6 Kits

In some embodiments, active ingredients provided herein are not administered to a patient at the same time or by the same route of administration. Therefore, encompassed are kits which, when used by the medical practitioner, can simplify the administration of appropriate amounts of active ingredients to a patient.

In one embodiment, provided herein is a kit which comprises a dosage form of a tumor cell engineered according to methods described herein and an immunomodulatory compound, or a pharmaceutically acceptable salt, solvate, stereoisomer, or prodrug thereof. Kits provided herein may further comprise additional active ingredients. Examples of the additional active ingredients include, but are not limited to, those disclosed herein (see, e.g., section 5.4.5).

Kits may further comprise devices that are used to administer the active ingredients. Examples of such devices include, but are not limited to, syringes, drip bags, patches, and inhalers.

Kits provided herein can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 6. EXAMPLES

Certain embodiments provided herein are illustrated by the following non-limiting examples.

6.1 Materials and Methods

6.1.1 Samples

Healthy donor leukopacks were obtained from Children's Hospital Boston. Normal bone marrow samples were purchased from AllCells, LLC (CA). MM patients' blood and bone marrow samples were obtained from Dana-Farber Cancer Institute, following informed consent approved by the institutional review boards.

6.1.2 Expansion of iNKT Cells

Dendritic cells (DCs) were generated from peripheral blood mononuclear cells (PBMCs) cultured with GM-CSF and IL-4. iNKT cells were first enriched from PBMCs by immunomagnetic isolation with Vα24 mAb (Immunotech, Marseille, France) and goat-anti-mouse IgG microbeads (Miltenyi Biotec, Auburn, Calif.), and then stimulated with irradiated (5000 rads) DCs, which were matured by LPS (100 ng/ml; Sigma-Aldrich) overnight in the presence of 100 ng/ml α-GalCer. Proliferating iNKT cells were further enriched by positive selection using Vβ11 magnetic beads (Immunotech). Cultures were then gradually expanded and restimulated every 7 to 10 days, containing 100 unit/ml IL-2. The purity of iNKT cells were monitored by quantifying Vα24⁺ Vβ11⁺ cells by flow cytometric analysis.

6.1.3 Detection of CD1d Expression and Preparation of CD1d-Transfected MM.1S Cell Line

Gene expression profiles of CD1d by multiple myeloma cell lines, CD138-positive normal plasma cells and primary myeloma cells were performed and analyzed as described in, e.g., Shammas et al., Blood, 108: 2804-2810 (2006). CD1d expression was also examined by flow cytometric analysis using CD1d-PE mAb (Pharmingen, La Jolla, Calif.). Multiple myeloma cell line MM.1S was transfected with a CD1d cDNA in the pSRα-neo expression vector and empty vector as described previously in, e.g. Exley et al., J Exp Med., 186: 109-120 (1997).

6.1.4 Immune Response of iNKT Cells and Primary Myeloma Cells

Resting iNKT cells were plated in 96-well plates (5×10⁴) with either medium alone or with 5×10⁴ of primary myeloma cells. αGalCer (100 ng/ml) was added as indicated. IL-2 was added at 10 units/ml. Activation of iNKT cells was detected after 48 hours incubation using double staining with anti-TCRVα24-FITC and anti-CD25-PC5 mAbs.

6.1.5 Characterization of iNKT Cells from Multiple Myeloma Patients and Effect of 1-Oxo-2-(2,6-dioxopiperidin-3-yl)-4-aminoisoindoline

1×10⁵ NKT cells from MM patients were plated in a 96-well plate with MM1S.CD1d cells or DCs at ratios of 2:1 and 4:1, respectively, with or without pre-loaded α-GalCer (100 ng/ml). 1-Oxo-2-(2,6-dioxopiperidin-3-yl)-4-aminoisoindoline (2 μM) was used as indicated, with DMSO as control. The supernatant was collected at 48 and 72 hours and the levels of IFN-γ, IL-2 and IL-4 were determined by ELISA (R&D Systems, Minneapolis, Minn.).

6.1.6 Statistical Analysis

Statistical analyses were performed by student t test. P<0.05 was considered significant.

6.2 Results

6.2.1 Establishment of Highly Purified iNKT Cell Lines

The frequency of iNKT cells in the PBMCs of healthy donors and multiple myeloma patients was determined using flow cytometric analysis double staining with a Vα24 and Vβ11 mAbs. The frequency of Vα24⁺ Vβ11⁺ cells in healthy donors was 0.064±0.030% (n=7), while reduced frequency was observed from advanced multiple myeloma patients (n=7) (0.01±0.008%, p<0.001).

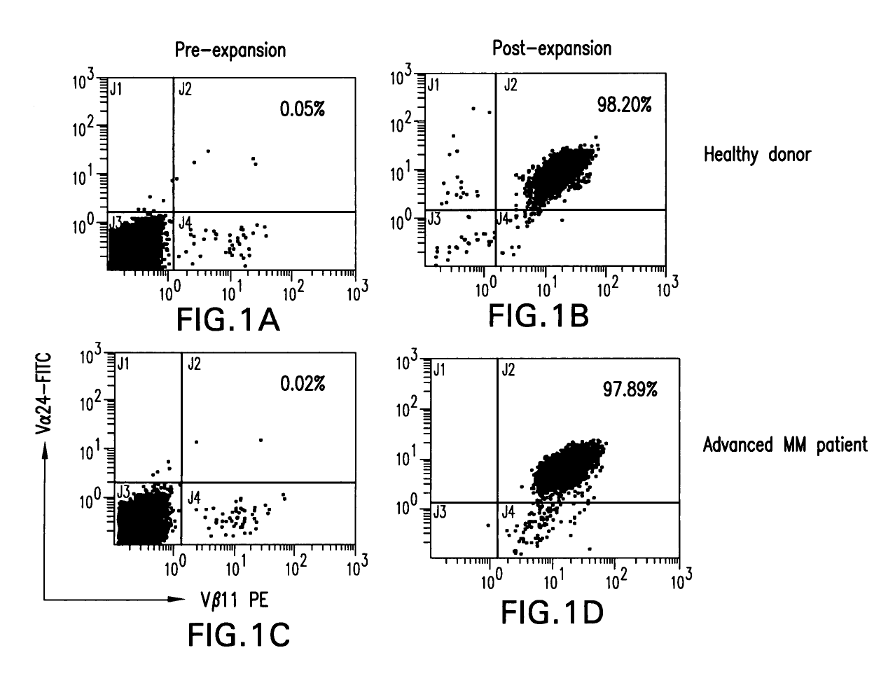

Next, highly purified iNKT cell lines from healthy donors, newly diagnosed patients, and advanced multiple myeloma patients were established. As shown in FIG. 1, flow cytometric analysis confirmed the purity of Vα24⁺ Vβ11⁺ cells is greater than 97 percent. The phenotype analysis showed the majority iNKT cells were CD4⁺ and CD4⁻ CD8⁻ cells. The expression of CD161 was variable (40.2˜99.4 percent), while none of iNKT cell lines expressed CD16 or CD94. A certain level CD56 expression was also observed (1.5˜17.2 percent). In general, greater than 10⁸ iNKT cells can be harvested in 6 weeks.

6.2.2 CD1d Expression on Multiple Myeloma Cell Lines and Primary Cells

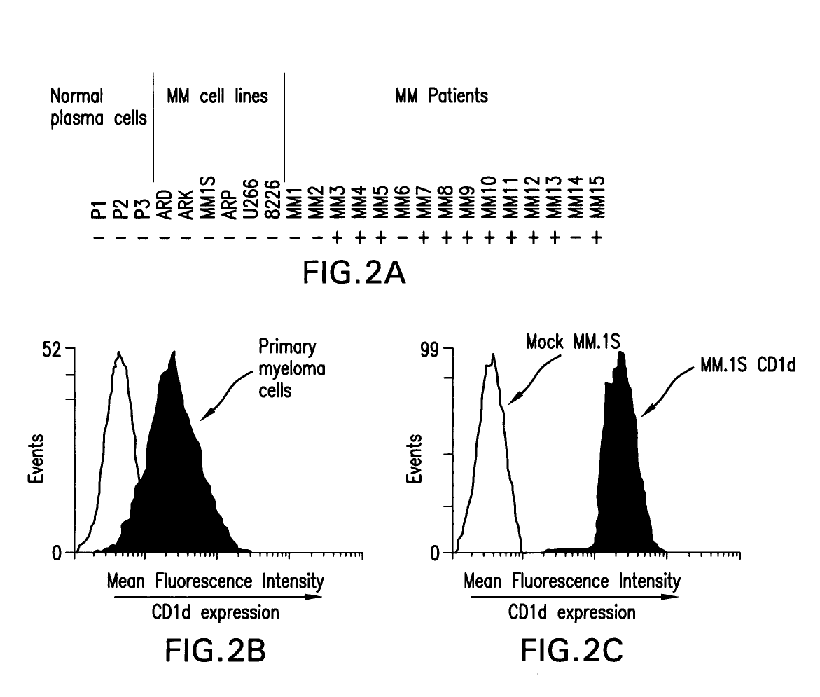

To evaluate potential in vivo interaction between iNKT cells and myeloma cells, the CD1d expression profile on primary myeloma cells and multiple myeloma cell lines were evaluated by gene expression profiling and flow cytometric analysis. The majority of primary multiple myeloma cells expressed high levels of CD1d (11 out of 15). In contrast, all 12 multiple myeloma cell lines tested (MM.1S, MM1R, ARD, ARK, ARP, PRMI8226, U266, OPM1, OPM2; CAG, 12PE and 28PE) showed no expression of CD1d. (FIG. 2 and data not shown).

6.2.3 Antigen Presentation by Primary Myeloma Cells

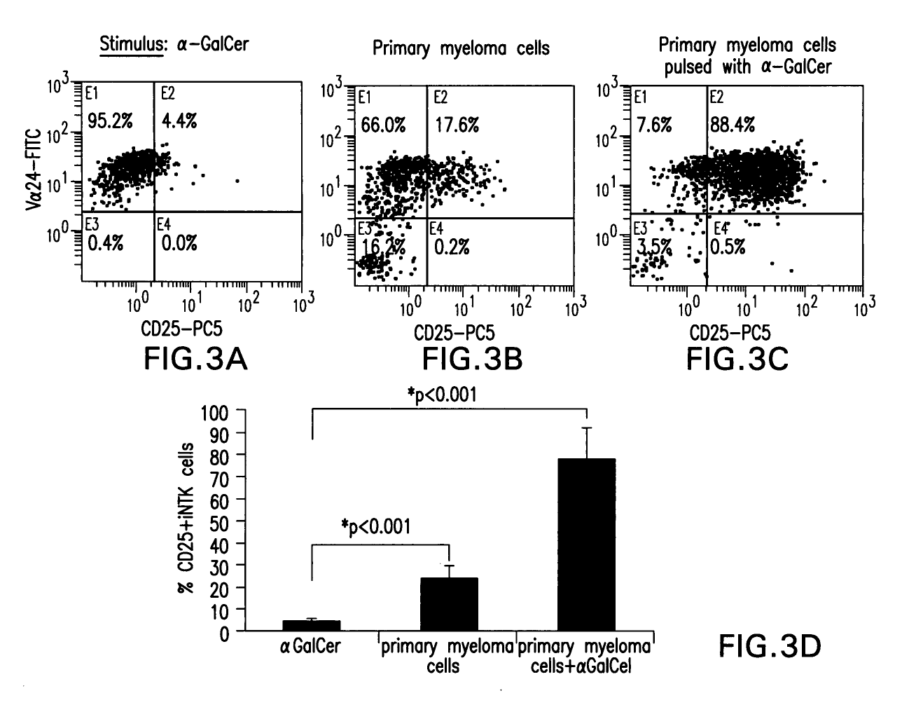

Whether iNKT cell lines from multiple myeloma patients could be activated by primary multiple myeloma cells was examined. As shown in FIG. 3, CD25 expression of iNKT cells was significantly increased in the presence of α-GalCer-pulsed primary myeloma cells (p<0.001). Moreover, iNKT cells co-cultured with primary myeloma cells, even in the absence of α-GalCer, also displayed significant increase in CD25 expression (p<0.001), indicating iNKT cells' reactivity towards endogenous ligands bound to CD1d.

6.2.4 Th-1 Cytokine Profile Exhibited by iNKT Cells

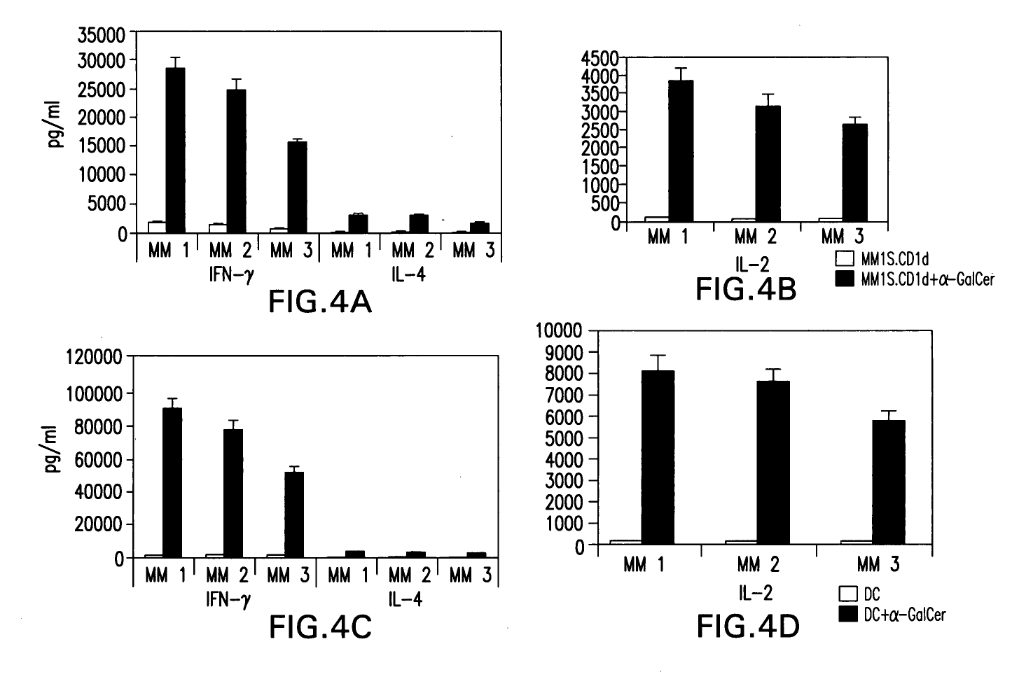

To further confirm function of α-GalCer-expanded iNKT cells from multiple myeloma patients and its CD1d-specific activation, cytokine profile using two types of antigen presenting cells, i.e., mock/CD1d-transfected MM.1S cell line and DCs, was evaluated. It was observed that iNKT cell lines stimulated by mock MM.1S cells, with or without α-GalCer, generated very low or undetectable levels of IL-2, IFN-γ and IL-4. When co-cultured with CD1d-transfected MM.1S cells (MM.1S.CD1d), iNKT cells secreted low, but higher, levels of those cytokines. As shown in FIG. 4A-4B, when stimulated with α-GalCer-pulsed MM.1S.CD1d cells, iNKT cells produced a high level of IFN-γ as well as IL-2, and a low level of IL-4. These results confirmed the CD1d-restricted activation of multiple myeloma iNKT cells. The ratio of IFN-γ and IL-4 indicates the establishment of Th-1-type iNKT cell lines. As shown in FIG. 4C-4D, these results were further confirmed by using α-GalCer-pulsed DCs, which showed even greater Th-1-biased cytokine responses. Thus, these results demonstrated that iNKT cell lines from advanced multiple myeloma patients are functional and respond to antigen presenting cells expressing CD1d with an antitumor cytokine profile.

6.2.5 Effect of 1-Oxo-2-(2,6-dioxopiperidin-3-yl)-4-aminoisoindoline on Th-1-Type Immune Response of iNKT Cell Lines

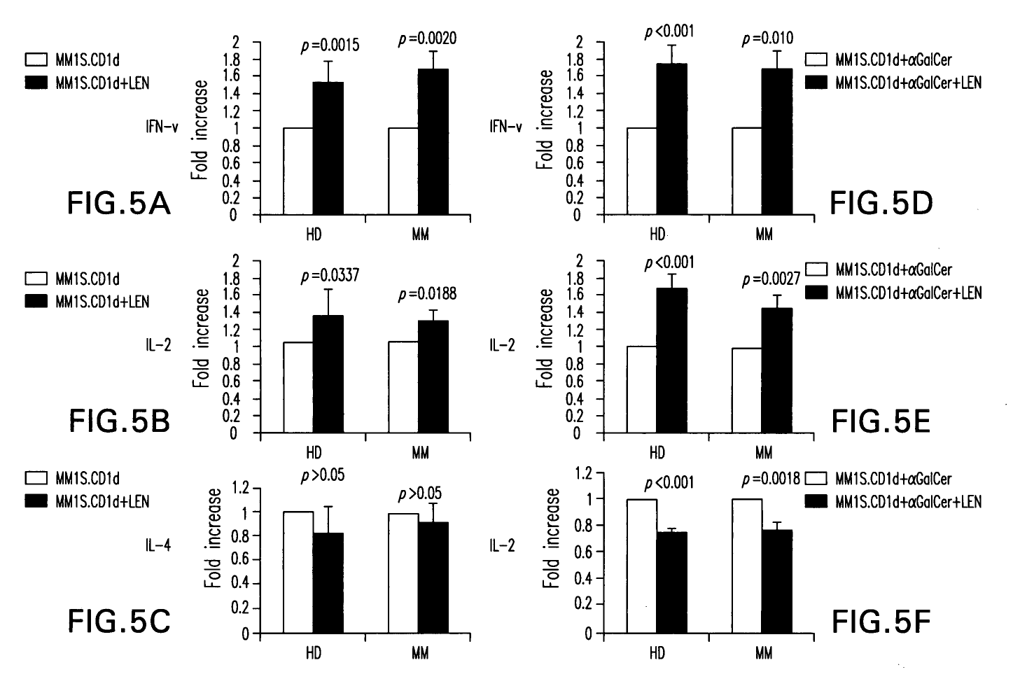

In an attempt to further augment iNKT cell antitumor effect, the effects of 1-oxo-2-(2,6-dioxopiperidin-3-yl)-4-aminoisoindoline, an immunomodulatory compound, on the antitumor immune response was examined. 1-Oxo-2-(2,6-dioxopiperidin-3-yl)-4-aminoisoindoline did not directly stimulate iNKT cells or iNKT cells co-cultured with mock MM.1S cells with or without of α-GalCer. However, as shown in FIG. 5, upon CD1d-restricted activation by α-GalCer-loaded MM1S.CD1d cells, 1-oxo-2-(2,6-dioxopiperidin-3-yl)-4-aminoisoindoline induced a significant increase in IFN-γ and IL-2 levels and decrease in IL-4 level, as compared to controls (MM1S.CD1d with or without α-GalCer). This phenomenon was also confirmed by using well-characterized CD1d-transfected C1R cells. Additionally, even in the absence of α-GalCer, 1-oxo-2-(2,6-dioxopiperidin-3-yl)-4-aminoisoindoline also increased production of IFN-γ and IL-2 by iNKT cell lines when co-cultured with MM1S.CD1d cells.

All of the references cited herein are incorporated by reference in their entirety. While the invention has been described with respect to the particular embodiments, it will be apparent to those skilled in the art that various changes and modifications may be made without departing from the spirit and scope of the invention as recited by the appended claims.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method of providing an iNKT cell responsive to multiple myeloma comprising:
(i) isolating an iNKT cell from a patient having multiple myeloma; and
(ii) incubating the isolated iNKT cell with an immunomodulatory compound.

2. The method of claim 1, further comprising contacting said iNKT cell with an multiple myeloma cell loaded with α-GalCer prior to step (ii).

3. The method of claim 1, wherein the iNKT cell is TCRVα24⁺ and TCRVβ11⁺.

4. The method of claim 1 wherein the multiple myeloma cell is CD1d-expressing primary myeloma cell.

5. The method of claim 4, wherein the primary myeloma cell is a multiple myeloma cell transfected with CD1d.

6. The method of claim 1, wherein the immunomodulatory compound is 1-oxo-2-(2,6-dioxopiperidin-3-yl)-4-aminoisoindoline or 1,3-dioxo-2-(2,6-dioxopiperidin-3-yl)-4 aminoisoindoline.

7. An iNKT cell, which is prepared according to the method of claim 1.

8. A pharmaceutical composition comprising the iNKT cell of claim 7.

9. A method of treating multiple myeloma in a patient comprising administering to said patient a therapeutically effective amount of the iNKT cell of claim 7.

10. A method of preparing a vaccine for multiple myeloma comprising:
(i) isolating an iNKT cell from a patient having multiple myeloma;
(ii) incubating said isolated iNKT cell with an immunomodulatory compound; and
(iii) irradiating the iNKT cell from step (ii).

11. The method of claim 10, further comprising contacting said iNKT cell with a multiple myeloma cell loaded with α-GalCer prior to step (ii).

12. A method of preventing multiple myeloma in a human comprising administering to a human the vaccine prepared using the method of claim 10.

13. A method of treating or preventing multiple myeloma in a patient comprising:
(i) isolating an iNKT cell from a patient having multiple myeloma;
(ii) irradiating the iNKT cell from step (i);
(iii) administering the iNKT cell to the patient; and
(iv) administering an immunomodulatory compound to the patient.

14. The method of claim 13, further comprising contacting said iNKT cell with a multiple myeloma cell loaded with α-GalCer prior to step (ii).
